(19) United States
(12) Patent Application Publication
(10) Pub. No.: US 2018/0250101 A1
(43) Pub. Date: Sep. 6, 2018

(54) IMPLANT FOR ANCHORING DENTAL PROSTHESIS

(71) Applicant: MDI DENTAL- UND IMPLANTATTECHNIK GMBH, Duisburg (DE)

(72) Inventors: Dirk-Rolf Gieselmann, Zurich (CH); Harald Ceschinski, Bochum (DE)

(21) Appl. No.: 15/972,403

(22) Filed: May 7, 2018

Related U.S. Application Data
(63) Continuation of application No. 12/310,959, filed on Nov. 23, 2009, now abandoned.

(30) Foreign Application Priority Data
Nov. 11, 2006 (DE) 102006053260.0
Jul. 20, 2016 (DE) 102006034092.2

(51) Int. Cl. A61C 8/00; C23C 14/34; C23C 14/06; A61L 27/30; C23C 14/02

(52) U.S. Cl. CPC A61C 8/0013; C23C 14/34; C23C 14/024; A61C 8/0012; A61L 27/306; C23C 14/0641

(57) ABSTRACT

An implant, in particular for anchoring dental prostheses, has a zirconium nitride (ZrN) coating covering in particular in the sections that come into contact with the soft tissue.

This is a continuation of U.S. patent application Ser. No. 12/310,959, filed Mar. 13, 2009 which is a national phase application of International Patent Application PCT/EP2007/006488 filed Jul. 20, 2007 which claims priority to German patent applications DE 10 2006 053 260.0, filed Nov. 11, 2006 and DE 10 2006 034 092.2, filed Jul. 20, 2006, all of which are hereby incorporated by reference herein.

The invention relates to an implant, in particular for anchoring dental prostheses.

### BACKGROUND

In modern medicine, implants are widely used to compensate congenital or acquired bodily defects. They are employed, for instance, in orthopedics for the fixation of fractures and, in the form of artificial joints or joint parts, they serve to compensate wear of the muscoskeletal system and they are extensively used in cardiology and vascular medicine. Furthermore, they are used in the stabilization of spinal columns, in the compensation of spinal damage as well as in osteosynthesis and traumatology. Finally, implants play a vital role as bone substitute and as devices for anchoring dental prostheses.

At the locus of implantation, the implants are bound to come into contact with is body tissue. This tissue may be, for instance, bone tissue or soft tissue, such as muscular tissue, connective tissue, epithelial tissue or mucosal tissue. It is desirable that the implant be not only compatible with the surrounding tissue, but that it may bond to and integrate with that tissue. Very often, the implant material is not equally compatible with all the surrounding types of tissue.

When a patient has lost one or more natural teeth, dental implants are today often used as a means to anchor dental prostheses, such as crowns, bridges or complete dentures.

Within the meaning of this invention, dental implants are supporting pillars that are anchored in the jawbone and have the function of an artificial tooth root. The implants are pillars that are firmly embedded (implanted) in the jawbone. They permit single teeth or bridges to be set in place without the need to alter adjacent healthy teeth.

The implantation process consists in securing the dental implant in a suitably prepared tooth root cavity by pressing or screwing it firmly in place. Two healing principles are known from the prior art, i.e. the “open” healing and the “closed” healing methods. In the “open” healing method, the visible upper end of the implant projects from the jawbone. In the “closed” healing method, in contrast, the implant terminates at the bone level. During the healing process, which usually takes three to six months, the bone tissue bonds directly to the implant. This phenomenon is known as “osseointegration”.

Implants designed for the “open” healing method comprise an enossal part and a gingival part. The enossal part is embedded in the jawbone. The gingival part projects from the jawbone and into the gums or gingiva. While the enossal part is subject to osseointegration, connective tissue and epithelial tissue will form around the gingival part.

With implants made of titanium—a material that is widely used for dental implants—complications in the osseointegration phase are rare. Titanium is well compatible with the bone tissue and the osseointegration process usually proceeds quickly and without any problem.

As for the compatibility of titanium with epithelial and gingival connective tissue, things are different. Here, the compatibility is significantly lower and the healing and integration process often lasts longer than would be desirable. Known complications are peri-implantitis and plaque accumulation. In fact, the integration of the gingival part of the implant with the gingiva should not take longer than the osseointegration.

It is known that epithelial and connective tissues are well compatible with ceramic materials and in particular with zirconium oxide. The use of zirconium oxide for implants—including dental implants—is known from the prior art. However, zirconium oxide has not, in all cases, proved equally suitable for dental prostheses as the widely used titanium implants. This may well be due to the extremely high pressures to which dental implants are subjected during chewing.

Given the extremely good compatibility of zirconium oxide ceramics with epithelial and gingival connective tissue, suggestions have been made to provide the gingival part of dental implants with a zirconium oxide sleeve cemented on to the titanium surface in order to make sure that the gingival tissue does not come into contact with the titanium metal of the implant proper. However, in this implant/sleeve combination, the cement bond has proved to be suboptimal, i.e. the sleeve tends to detach itself from the titanium core after only a short period of time.

Dental implants that are provided with a zirconium sleeve in the gingival area are described in WO 2003/013385 A.

### SUMMARY OF THE INVENTION

It is an object of the present invention to provide implants which offer further improvements in the good compatibility or osseointegration properties of metal implants and which are optimized in terms of integration with soft tissue and durability.

The present invention provides implants of the type mentioned at the outset, which implants are provided with a coating of stabilized zirconium nitride and/or zirconium oxide.

The implants according to the invention can be used in almost any medical area. They are described in detail below, using dental implants as examples. The soft tissue coming into contact with the implants is, in this case, the gingiva which corresponds with the gingival part of the implant. In the case of other implants, such soft tissue may be connective tissue, muscular tissue, epithelial tissue or a form of mucosal tissue.

On dental implants, the coating of the gingival part avoids the necessity of a cement layer of the type required for attaching the sleeve according to WO 2003/013385.

Metals can be coated—with good adherence within the coating and between the coating and the metal—using in particular cathode sputtering. In the cathode sputtering process, material is removed from a cathode (target) and precipitates on a substrate. The coating process takes place in a vacuum chamber in the presence of an inert gas such as argon (gas flow sputtering, GFS). Reactive sputtering allows a further gas to be introduced, which reacts with the ions removed from the target. The coatings obtained with this process are ceramic in nature if oxygen or nitrogen is introduced.

For coating the gingival part of the implant, the invention preferably uses this type of reactive gas sputtering process, which takes place in a vacuum chamber in the presence of nitrogen or oxygen. In this context, reference is made to DE 199 58 643 C1 and the technologies described therein for coating turbine blades with zirconium oxide. The target is metallic zirconium alloyed with up to 10% w/w of yttrium. The implant or implants is/are placed on a heated substrate holder.

To prevent the coating from depositing also on the enossal part of the implant, the latter is shielded in the known fashion. The same applies to other implants that are to be coated only in part.

A metal oxide may be admixed to the zirconium oxide in the known fashion. Such polycrystalline “stabilized” zirconium oxide contains, for example, up to 10% w/w of yttrium oxide (Y₂O₃) and also, where expedient, aluminum oxide (Al₂O₃), magnesium oxide (MgO), calcium oxide (CaO) or several of these oxides The stabilized zirconium oxide coating can be produced, for example, by simultaneous cathode sputtering of zirconium oxide and yttrium in the presence of oxygen. Where the term “zirconium” is used hereinafter, it may—as a metal or a nitride or an oxide—contain up to 10% w/w of yttrium, usually 5 to 10% w/w.

Independently of the foregoing, other sputtering processes are also suitable for producing the desired coating, e.g. DC sputtering, HF sputtering, magnetron sputtering and ion beam sputtering, including electron beam evaporation, the preferred process being GFS sputtering.

An implant coating of zirconium nitride is yellow to gold in color and of such a nature that even thin coatings can mask the grey color of the metal in the desired manner. Such a zirconium nitride coating usually has a film thickness of between 1 and 10 microns, depending on the desired color depth and the thickness of the extremely hard zirconium nitride coating.

Physiologically speaking, zirconium not only has the benefit of extreme hardness, which increases the coating's abrasion resistance, but it also exhibits extremely good physiological compatibility. Zirconium nitride—like zirconium oxide—promotes the bonding to the gingival tissue. What is more, both zirconium nitride and zirconium oxide pick up the texture produced by the metal working operation and impart it to the surface, which means that the implants, even in coated condition, have a transversely fluted texture which shows on the surface. Colonialization with gingival cells takes place preferably in the flutes, i.e. in transverse direction to the direction in which loads are exerted on the implant, which provides additional strength.

To further improve the bond between the coating and the metal of the implant, it may be expedient to apply a base coating and/or intermediate coating, for example by oxidizing the metal surface (by converting titanium into titanium oxide), by applying a zirconium base coating or by applying an intermediate coating of zirconium nitride or aluminum oxide (Al₂O₃). These base and intermediate coatings, too, can be produced by cathode sputtering.

It is possible, for example, to first apply a zirconium base coating of 0.5 to 2 microns to the titanium surface cleaned using the GFS process. Subsequently, an intermediate coating of zirconium nitride (ZrN) with a film thickness of between 1 and 10 microns is applied. On this coating, a zirconium oxide covering coating is applied. All coatings can be applied in one process comprising glow-discharge treatment in the presence of argon, application of the base coating by sputtering with a zirconium target in argon, application of the nitride coating by sputtering with a zirconium target in argon and nitrogen, and application of the oxide coating by sputtering with zirconium target in argon and oxygen. The substrate is heated to an elevated temperature of up to 400° C. The bias tension may be as high as 200 volts, depending on the process step, and the bias frequency may be as high as 200 kHz, depending on the process step as well.

Generally, the zirconium oxide coating has a film thickness of between 2 and 50 microns, and in particular between 5 and 15 microns. For the base coating, if required; a film thickness of approx. 1 micron is usually sufficient.

A typical dental implant as shown in FIG. 3 is provided, on the cleaned titanium surface, with—for example—a zirconium film of 0.5 to 2 microns, especially 1 to 1.5 microns, a zirconium nitride film of 1 to 10 microns, especially approx. 4 to 5 microns, and a zirconium oxide film of 10 to 50 microns, especially 10 to 20 microns, particularly preferably approx. 15 microns.

A coating built up in this fashion provides a sufficiently hard surface of acceptable color and with excellent healing and integration properties, whereby the affinity of the mucosal tissue for the zirconium oxide surface counteracts parondontosis—a highly feared risk in dental prosthetics.

The coating of the implant generally covers its gingival section, i.e. all parts that come into contact with the gingiva. The coating may also extend to the upper surfaces of the implant and, depending on the circumstances, also to parts that project from the gingiva into the oral cavity and serve to secure a crown, for instance.

In the case of implants that are made up of several parts, such as the implants for the “closed” healing method mentioned above, which terminate at the bone level, it is clear that the gingival abutment attached to this part of the implant must be coated with stabilized zirconium oxide. Further parts may be coated as well, such as pins or tops which are fixed to the implant and serve to secure the prosthesis proper. Coating such pins or tops has the benefit of improving the visual appearance. As the mounted crowns are mostly translucent, the metal cores tend to shine through them. This casts a visually unappealing dark shadow through the dental prosthesis. Coating these pins or tops provides an optical screen that enhances the visual appearance of the entire dental prosthesis.

The implants according to the invention have still further advantages. They avoid or reduce peri-implantitis, which often occurs at dental implants and may ultimately lead to the loss of the implant. Reduction of plaque accumulation is a further advantage.

The above applies also to implants that are intended for the non-dental area of medicine. Here, too, the implants may be coated in whole or in part, the important thing being that those parts are coated that come into contact with soft tissue.

The implants coated according to the invention are optimally adapted in terms of healing/integration, especially soft tissue integration, and have significantly better tissue compatibility. This contributes to a shorter healing/integration period and higher load resistance compared to conventional implants.

### BRIEF DESCRIPTION OF THE DRAWINGS

The invention is explained in detail based on the attached Figures.

FIG. 1 shows an implant 1 as defined by the invention, whose enossal section 2 is screwed into the jawbone by means of the thread 3;

FIG. 2 is a schematic of the coating structure including the rotating substrate holding plate.

A typical dental implant as shown in FIG. 3 is provided, on the cleaned titanium surface, with a zirconium film.

### DETAILED DESCRIPTION

The invention is explained in detail based on the attached Figures.

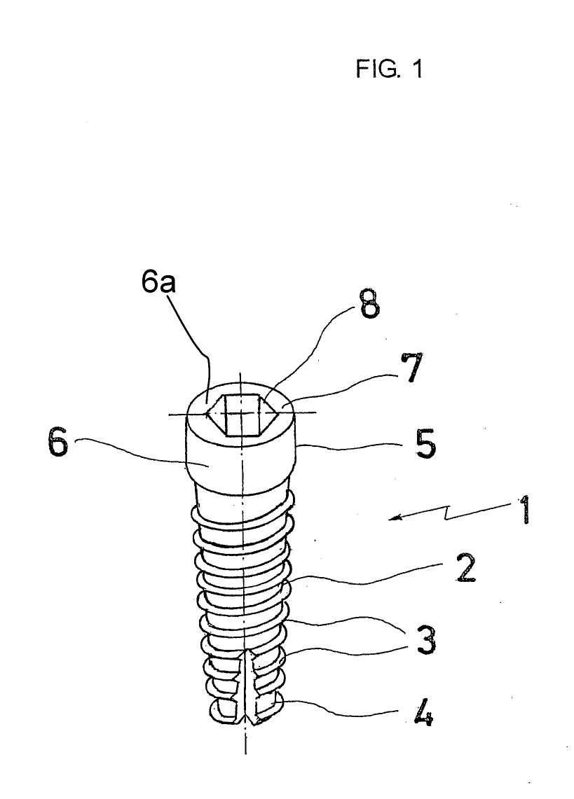

FIG. 1 shows an implant 1 as defined by the invention, whose enossal section 2 is screwed into the jawbone by means of the thread 3. The root section 4 is provided with notches in the usual fashion to facilitate osseointegration.

The gingival section 5, which follows the enossal section 2, is coated with a thin film of zirconium ceramic material 6, which coating may be confined to the flank of the implant or it may extend at 6a further to cover also the top part 7. The top area 7, which terminates at the gingiva, has a hexagonal opening 8 which can be used, on the one hand, to screw the implant into the jawbone and, on the other hand, to accept a pin that carries the dental prosthesis proper.

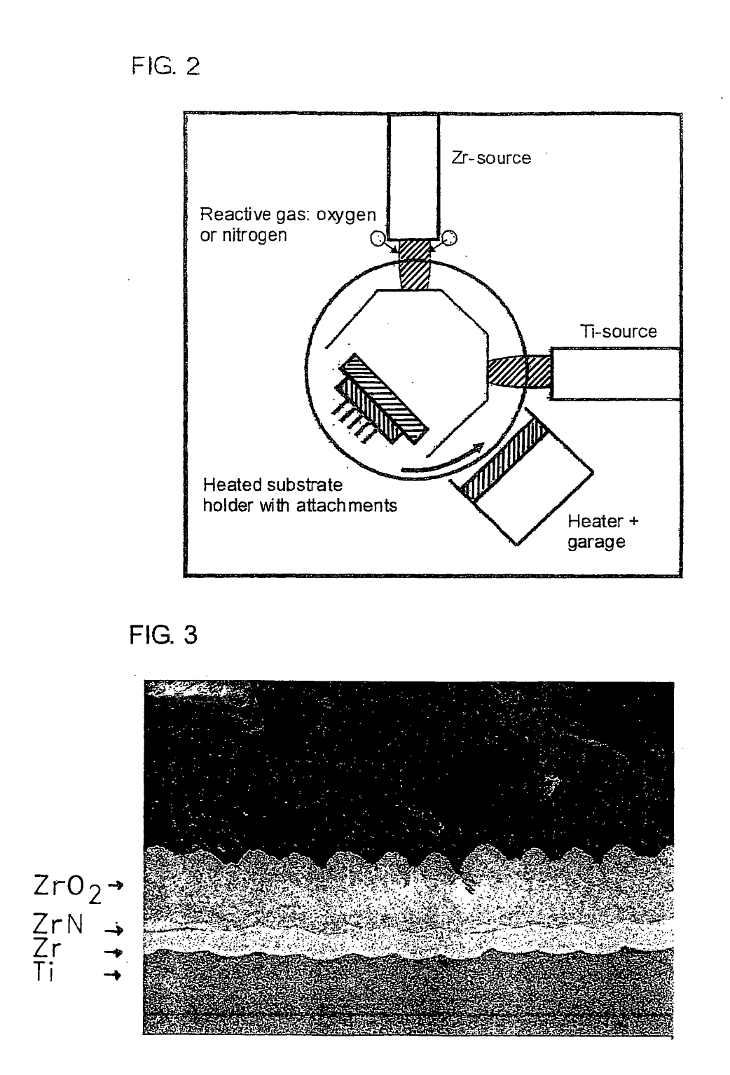

FIG. 2 is a schematic of the coating structure including the rotating substrate holding plate. The coating chamber is a polygon vacuum system having a chamber volume of 2001, which is equipped with a horizontally operating GFS linear source provided with metallic targets of zirconium yttrium (92.2:7.8% w/w). In the reactive process, yttrium-part-stabilized zirconium oxide is ejected while oxygen is introduced into the process; zirconium nitride (+yttrium nitride) is ejected when nitrogen is added. A second sputter source (Ti source) allows the deposition of an additional titanium adherence film.

Inlets for the reactive gases—oxygen and nitrogen—are provided near the Zr source.

A substrate holding plate, which is heated from the rear face, serves to hold the substrate. A ceramic radiation heater permits the substrate to be heated to temperatures of up to 400.degree. C., which are monitored by means of a thermocouple placed in contact with the holding plate.

The gases used in the process are argon, which serves for material transfer, as well as oxygen and nitrogen which are used as reactive gases, both of which are required to have a very high purity of at least 99.99%.

For the application of the coatings, the implants, placed on the substrate holder, are first glow-discharge-treated in the presence of argon to remove surface dirt and oxide layers. Subsequently a zirconium base coating is deposited in the presence of argon, then follows the application of a zirconium nitride intermediate coating while additional nitrogen is introduced into the process. Finally, the zirconium covering coating can be deposited in the presence of oxygen (the nitrogen supply is switched off).

It goes without saying that the zirconium oxide covering coating can be dispensed with where extremely hard surfaces are required. In this case, a zirconium nitride covering coating can be used instead. For such a zirconium nitride covering coating, a film thickness of about 10 microns is sufficient, but it may be applied also in thicker films of up to 20 microns.

What is claimed is:

1: An implant, in particular for anchoring dental prostheses, comprising a zirconium nitride (ZrN) and/or a zirconium oxide (ZrO₂) coating.

2: An implant according to claim 1, wherein the zirconium oxide is stabilized by yttrium oxide and/or aluminum oxide.

3: An implant according to claim 1, wherein the zirconium oxide contains up to 10% w/w of yttrium oxide.

4: An implant according to claim 1, wherein the zirconium nitride and/or the zirconium oxide coating can be obtained by cathode sputtering.

5: An implant according to claim 4, wherein the zirconium oxide coating can be obtained by reactive gas flow sputtering performed in a vacuum chamber in the presence of oxygen.

6: An implant according to claim 1, wherein the film thickness of the zirconium oxide coating is 2 to 50 microns.

7: Implant according to claim 6, wherein the film thickness is 5 to 15 microns.

8: An implant according to claim 1, wherein it is provided with an adherence-promoting base coating.

9: An implant according to claim 8, wherein the base coating consists of zirconium, zirconium nitride, titanium oxide (TiO₂) and/or aluminum oxide (Al₂O₃).

10: An implant according to claim 1, wherein it is provided with a hard intermediate coating.

11: An implant according to claim 10, wherein the intermediate coating consists of zirconium nitride.

12: An implant according to claim 1, wherein the coating covers at least the sections that come into contact with the soft tissue (gingiva).

13: An implant according to claim 11 comprising an enossal section and a gingival section, comprising a zirconium base coating, a zirconium nitride intermediate coating and a zirconium oxide (ZrO₂) covering coating applied at least in the gingival section.

14: An implant according to claim 13, wherein the base coating has a film thickness of at least 0.5 to 2 microns.

15: An implant according to claim 13, wherein the intermediate coating has a film thickness of at least 1 to 10 microns.

16: An implant according to claim 8, further comprising a zirconium base coating and a zirconium nitride (ZrN) covering coating.

17: An implant according to claim 1, wherein the coating covers the top part of the implant.

18: An implant according to claim 1, wherein it consists of titanium or a titanium alloy.
